SEQUENCE LISTING (1 sequence)

SEQ ID NO: 1 - protein, 100 aa, Homo sapiens
MADGSSDAAREPRPAPAPIRRRSSNYRAYATEPHAKKKSKISASRKLQLKTLLLQIAKQELEREAEERRGEKGRALSTRCQPLELTGLGFAELQDLCRQL

(19) United States
(12) Patent Application Publication
(10) Pub. No.: US 2026/0235621 A1
(43) Pub. Date: Aug. 13, 2026

(54) METHODS OF IDENTIFYING MACROTROPONIN IN BIOLOGICAL SAMPLES

(71) Applicant: ABBOTT LABORATORIS, Abbott Park, IL (US)

(72) Inventors: John C. PROSTKO, Abbott Park, IL (US); Matthew Matlas, Abbott Park, IL (US); Edwin C. Frias, Abbott Park, IL (US)

(21) Appl. No.: 19/126,377

(22) PCT Filed: Nov. 8, 2023
(86) PCT No.: PCT/US2023/036990

§ 371 Date: May 1, 2025

Related U.S. Application Data
(60) Provisional application No. 63/424,230, filed on Nov. 10, 2022.

(51) Int. Cl. G01N 33/68

(52) U.S. Cl. CPC G01N 33/6878; G01N 33/6887; G01N 2333/4712

(57) ABSTRACT

The present disclosure provides methods for determining the presence macrotroponin in biological samples obtained from subjects.

### RELATED APPLICATION INFORMATION

This application is a national stage application of PCT/US2023/036990, filed Nov. 8, 2023, which claims priority to U.S. Application No. 63/424,230 filed on Nov. 10, 2022, the contents of which are herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,026 Byte XML file named “ABBTL_39607_601_SequenceListing.xml,” created on Apr. 29, 2025.

### FIELD

The present disclosure relates to methods for identifying the presence of macrotroponin and/or differentiating macrotroponin from troponin in one or more biological samples obtained from a subject.

### BACKGROUND

Cardiac troponins, such as cardiac troponin I (cTnI) and cardiac troponin T (cTnT), are biomarkers of myocardial injury. High-sensitivity troponin assays allow for the measurement of very low levels of cTnI and cTnT in biological samples with excellent precision. However, it is known in the art that in some instances, these assays can report elevated cTnI and cTnT levels which that do not appear consistent with the clinical picture (See, Warner et al., “High incidence of macrotroponin I with a high-sensitivity troponin I assay,” Clin. Chem. Lab Med. 1:54(11):1821-1289 (November 2016); Akhtar, et al., “False-positive troponin elevation due to an immunoglobulin-G-cardiac troponin T complex: a case report,” European Heart Journal, 4:1-5 (2020)).

It has been determined that some cases involving elevated cTnI and cTnT levels reported using high-sensitivity troponin assays are the result of macrotroponin, a high molecular weight complex that forms between endogenous cardiac troponin autoantibodies and circulating cardiac troponins. Unfortunately, identifying the presence of macrotroponin in biological samples is not simple and typically involves labor intensive, manual methods, such as polyethylene glycol (PEG) precipitation, protein A chromatography, and/or size exclusion chromatography. Therefore, a need exists for less labor intensive, automated methods for detecting the presence of macrotroponin in biological samples.

### SUMMARY

In one embodiment, the present disclosure relates to a method for identifying the presence of macrotroponin in a biological sample obtained from a subject. In some aspects, the method comprises the steps of:
a) providing a first amount of cardiac troponin I (cTnI) obtained from an assay on a biological sample obtained from a subject (“assay result 1”), wherein the assay employs at least one antibody or antibody fragment that recognizes or specifically binds to an epitope between amino acids 1 to 50 or 137 to 209 on cTnI;
b) providing a second amount of cardiac troponin I (cTnI) obtained from an assay from the same biological sample from the subject (“assay result 2”), wherein the assay employs at least one antibody or antibody fragment that recognizes or specifically binds to an epitope between amino acids 51 to 136 on cTnI;
c) determining a percentage difference of cTnI in the sample using the formula: (1-(assay result 2/assay result 1))×100%; and
d) determining that macrotroponin is present in the sample if the percentage difference determined in step c) is less than about −15% or greater than about 45%.

In some aspects, in the above method, the epitope between amino acids 1 to 50 or 137 to 209 has a length of 4 to 50 amino acids. In other aspects of the above method, the epitope between amino acids 51 to 136 has a length of 4 to 20 amino acids.

Additionally, in another aspect, the method employed to provide assay result 1 employs at least one antibody or antibody fragment that recognizes or specifically binds to epitopes 24-40 on cTnI.

In yet another aspect of the above method, the assay employed to provide assay result 2 employs at least one antibody or antibody fragment that recognizes or specifically binds to epitopes 87 to 91 on cTnI.

In still yet another aspect of the above method, the volume of biological sample used in the method is less than about 500 μL, less than about 400 μL, less than about 300 μL, less than about 200 μL, less than about 100 μL, less than about 90 μL, less than about 80 μL, less than about 70 μL, less than about 60 μL, less than about 50 μL, less than about 40 μL, less than about 30 μL, less than about 20 μL, or less than about 10 μL.

In still yet another aspect, any assay used in the above method is a high-sensitivity cTnI assay. Moreover, in some aspects, the assay is an immunoassay, a clinical chemistry assay, a lateral flow assay, or any combinations thereof. In still yet another aspect, the assay is a point-of-care assay or a single molecule detection assay.

In still a further aspect, the biological sample used in the method is whole blood, capillary blood, fingerstick blood, plasma, serum, urine, or any combinations thereof. In yet another aspect of the above method, the subject is a human.

In still a further aspect of the above method, macrotroponin is identified as being present in the sample if the percentage difference determined in step c) is less than −15% or greater than about 45%.

In still yet another aspect, the assay result 1 is between about 10 ng/L to about 600 ng/L. In still yet another aspect, the assay result 1 is between about 10 ng/L to about 100 n/L.

In another embodiment, the present disclosure relates to a method for identifying the presence of macrotroponin in a biological sample obtained from a subject. the method comprises the steps of:
a) providing a first amount of cardiac troponin I (cTnI) obtained from an assay on a biological sample obtained from a subject (“assay result 1”), wherein the assay employs at least one antibody or antibody fragment that recognizes or specifically binds to an epitope between amino acids 1 to 50 or 137 to 209 on cTnI;
b) providing a second amount of cardiac troponin I (cTnI) obtained from an assay from the same biological sample from the subject (“assay result 2”), wherein the assay employs at least one antibody or antibody fragment that recognizes or specifically binds to an epitope between amino acids 51 to 136 on cTnI;
c) determining a ratio of cTnI in the sample using the formula: 1−(assay result 2/assay result 1); and
d) determining that macrotroponin is present in the sample if the ratio determined in step c) is less than about −0.15 or greater than about 0.45.

In some aspects, in the above method, the epitope between amino acids 1 to 50 or 137 to 209 has a length of 4 to 50 amino acids. In other aspects of the above method, the epitope between amino acids 51 to 136 has a length of 4 to 20 amino acids.

Additionally, in another aspect, the method employed to provide assay result 1 employs at least one antibody or antibody fragment that recognizes or specifically binds to epitopes 24-40 on cTnI. In yet another aspect of the above method, the assay employed to provide assay result 2 employs at least one antibody or antibody fragment that recognizes or specifically binds to epitopes 87 to 91 on cTnI.

In still yet another aspect of the above method, the volume of biological sample used in the method is less than about 500 μL, less than about 400 μL, less than about 300 μL, less than about 200 μL, less than about 100 μL, less than about 90 μL, less than about 80 μL, less than about 70 μL, less than about 60 μL, less than about 50 μL, less than about 40 μL, less than about 30 μL, less than about 20 μL, or less than about 10 μL.

In still yet another aspect, any assay used in the above method is a high-sensitivity cTnI assay. Moreover, in some aspects, the assay is an immunoassay, a clinical chemistry assay, a lateral flow assay, or any combinations thereof. In still yet another aspect, the assay is a point-of-care assay or a single molecule detection assay.

In still a further aspect, the biological sample used in the method is whole blood, capillary blood, fingerstick blood, plasma, serum, urine, or any combinations thereof.

In yet another aspect of the above method, the subject is a human.

In still a further aspect of the above method, macrotroponin is identified as being present in the sample if the percentage difference determined in step c) is less than −0.20 or greater than about 0.50. In still yet another aspect, the assay result 1 is between about 10 ng/L to about 600 ng/L. In still yet another aspect, the assay result 1 is between about 10 ng/L to about 100 n/L.

Other embodiments and embodiments of the disclosure will be apparent in light of the following detailed description and related figures.

### BRIEF DESCRIPTION OF THE FIGURES

FIG. 1A shows epitopes that could be used for the capture and/or detection of troponin, amino acids 1-50 and/or 137 to 209 (not shown) on cTnI that are not recognized by macrotroponin whereas amino acids 51-136 that could be used for the capture and/or detection of troponin are also recognized by macrotroponin.

FIG. 1B shows an embodiment of an assay for cTnI which employs capture and detection specific binding partners (e.g., antibodies) that recognize and/or specifically bind to epitopes in areas other than between amino acids 51-136 and which generate a detectable signal if any troponin is present in the biological sample, regardless of any interference by macrotroponin at amino acids 51-136.

FIG. 2 shows an embodiment of an assay for cTnI which employs one or more capture and/or detection specific binding partners (e.g., antibodies) that recognize and/or specifically bind to epitopes between amino acid 51-136 and generate a reduced detectable signal if troponin and macrotroponin are each present in the biological sample, thus providing a falsely depressed result in contrast to Assay 1 which is not affected by any interference by macrotroponin.

FIG. 3 shows an embodiment for an assay for cTnI which employs one or more capture specific binding partners (e.g., antibodies) that recognize and/or specifically bind to epitopes between amino acid 51-136 and generate a reduced detectable signal if troponin and macrotroponin are each present in the biological sample, thus providing a falsely depressed result in contrast to Assay 1.

### DETAILED DESCRIPTION

Disclosed herein are methods for identifying the presence of macrotroponin and/or differentiating macrotroponin from troponin in one or more biological samples obtained from a subject. Previous methods for identifying macrotroponin in biological samples used manual, labor intensive techniques, such as PEG precipitation, protein A chromatography, and/or size exclusion chromatography. Described herein are automated methods for identifying macrotroponin and/or differentiating macrotroponin from troponin in biological samples which demonstrate agreement with the manual, labor intensive techniques known in the art thereby avoiding the need for multiple types of instrument systems in a testing facility or lab.

### I. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

An “antibody” or “antibodies” as used herein, refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-troponin antibody or a troponin antibody).

An “antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

A “high-sensitivity cardiac troponin assay” or “high-sensitivity cTn assay” as used interchangeably herein, refers to an assay (e.g., such as an immunoassay, a clinical chemistry assay, a lateral flow assay, a single molecule detection assay, a point-of-care assay, etc.) that is capable of detecting a cardiac troponin (such as cTnI or cTnT) with a coefficient of variation/variance <10% at or below the 99th percentile value in a population of interest (e.g., normal or healthy individuals). To be classified as a high-sensitivity assay, concentrations below the 99% percentile should be detectable above the assay's limit of detection for >50% of healthy individuals in the population of interest.

Examples of high-sensitivity cTnI assays include the Abbott ARCHITECT® STAT High-Sensitivity Troponin I assay, the Beckman ACCESS High-Sensitivity Troponin I assay, the Mitsubishi PATHFAST™ Troponin I assay, the Radiometer AQT90 FLEX Troponin I assay, the Singulex Erenna® Troponin I assay, the Siemens Dimension Vista® High-Sensitivity Troponin I assay, and the Siemens ADVIA Centaur High-Sensitivity Troponin I assay. An example of a high-sensitivity cTnT assay that can be used is the Roche Elecsys® Gen 5 STAT High-Sensitivity Troponin T assay.

“Cardiac troponin I” or “cTnI” as used interchangeably herein, refers to the I subunit of troponin. Mature cTnI comprises 209 amino acids. Cardiac troponin I has a molecular mass of 24 kDa and has five domains: N-terminal domain, IT-arm, inhibitory domain, regulatory domain, and C-terminal domain.

“Cardiac troponin T” or “cTnT” as used interchangeably herein, refers to the T subunit of troponin. Mature cTnI comprises 287 amino acids. Cardiac troponin T contains a N-terminal variable domain, conservative central residues, and a C-terminal domain.

A “point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Such point-of-care devices can also include portable, desktop sized devices. Examples of point-of-care devices include those produced by Abbott Laboratories (Abbott Park, IL) (e.g., i-STAT®, i-STAT® Alinity, ID Now®), Universal Biosensors (Rowville, Australia) (see US 2006/0134713), Axis-Shield PoC AS (Oslo, Norway) and Clinical Lab Products (Los Angeles, USA). As such, in some embodiments, the processing system further comprises a hand-held or point-of-care testing device. A point-of-care device typically uses one or more point-of-care assays.

A “sample”, “test sample”, or “biological sample” as used interchangeably herein, can include, but is not necessarily limited to, bodily fluids such as blood-related samples (e.g., whole blood, capillary blood, fingerstick blood, serum, plasma, and other blood-derived samples), urine, interstitial fluid, cerebral spinal fluid, bronchoalveolar lavage, and the like. Another example of a biological sample is a tissue sample. A biological sample may be fresh or stored (e.g., blood or blood fraction stored in a blood bank). The biological sample may be a bodily fluid expressly obtained for the assays of this invention or a bodily fluid obtained for another purpose which can be sub-sampled for the assays of this invention. In certain embodiments, the biological sample is whole blood. Whole blood may be obtained from the subject using standard clinical procedures. In other embodiments, the biological sample is plasma. Plasma may be obtained from whole blood samples by known means, including but not limited to, centrifugation (e.g., of anti-coagulated blood), membrane- or filter-based separation, agglutination-based plasma separation, acoustic force, and microfluidics. Such process provides a buffy coat of white cell components and a supernatant of the plasma. In certain embodiments, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. In another embodiment, the sample is urine. The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

In some aspects, the samples used to provide a first assay result described herein are blood (e.g., whole blood, capillary blood, fingerstick blood, etc.) serum, or plasma samples. In some embodiments, the samples used to provide a second assay result described herein comprise blood (e.g., whole blood, capillary blood, fingerstick blood, etc.), serum, or plasma samples.

The sample can be obtained using techniques known to those skilled in the art, and the sample may be used directly as obtained from the source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

The samples may be obtained in a medical facility, e.g., at an Emergency Room, urgent care clinic, walk-in clinic, a long-term care facility, or another appropriate site of medical practice. The sample may be obtained in a home or residential setting (e.g., a senior living or hospice setting), at the site of the suspected myocardial infarction, or during transportation to a medical facility (e.g., ambulance).

“Solid phase” or “solid support” as used interchangeably herein, refers to any material that can be used to attach and/or attract and immobilize (1) one or more capture agents (e.g., a capture antibody) or capture specific binding partners, or (2) one or more detection agents (e.g., a detection antibody) or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody or antibody fragment, a protein, or a peptide with a second chemical species, wherein the interaction is dependent upon the presence of a particular structure (e.g., an antigenic determinant or epitope) on the chemical species; for example, an antibody or antibody fragment recognizes and binds to a specific protein structure rather than to proteins generally. If an antibody is specific for epitope “A”, the presence of a molecule containing epitope “A” (or free, unlabeled “A”), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

A “subject” or “patient” may be human or non-human and may include, for example, animal strains or species used as “model systems” for research purposes, such a mouse model as described herein. Likewise, subject may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### II. Methods of Identifying Macrotroponin and/or Differentiating Macrotroponin from Troponin in Biological Samples

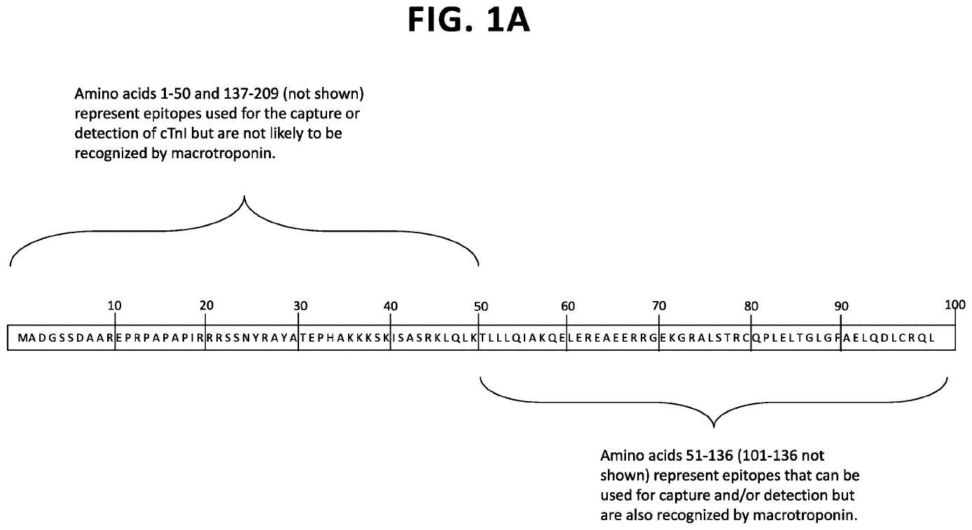

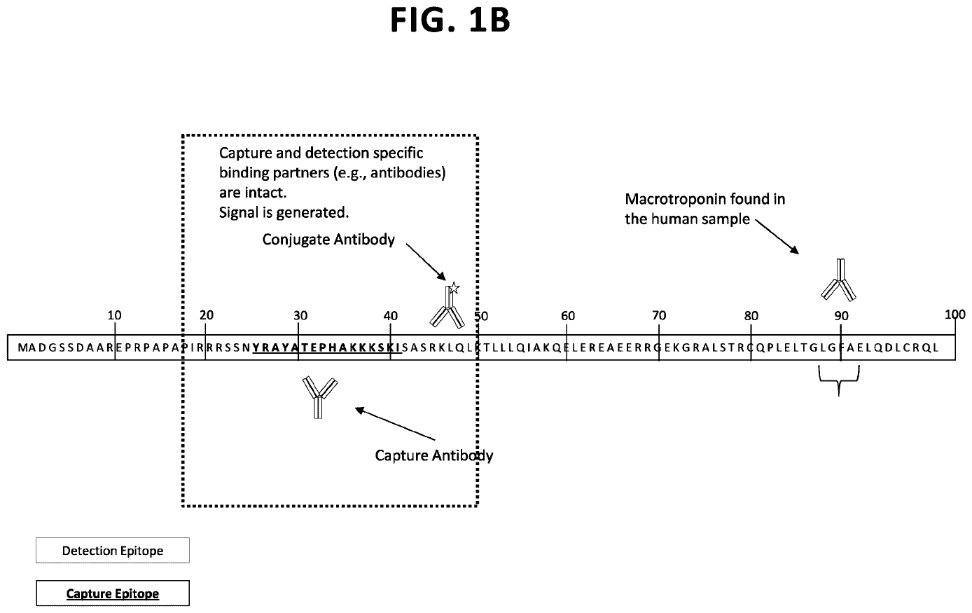

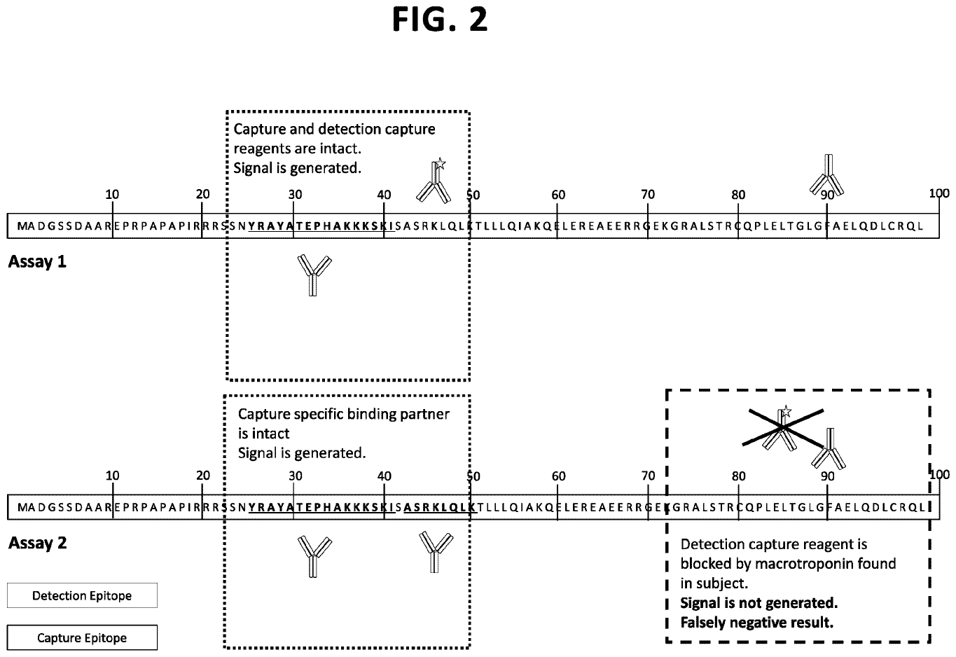

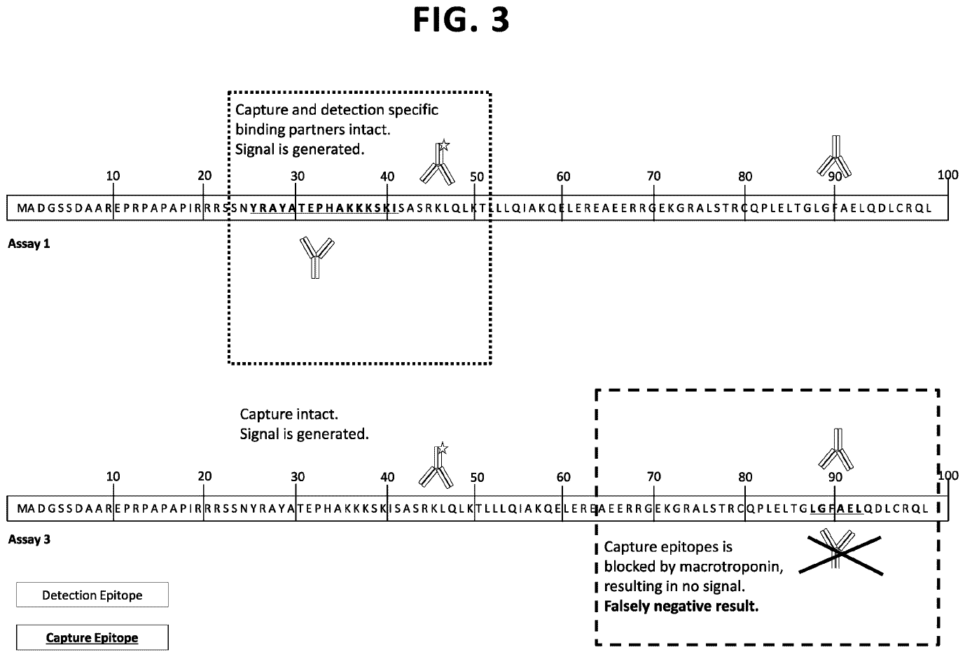

In one embodiment, the present disclosure relates to methods of identifying macrotroponin in one or more biological samples obtained from a subject. Additionally, or alternatively, in another embodiment, the present disclosure relates to methods of differentiating macrotroponin from troponin in one or more biological samples obtained from a subject. More specifically, the methods of the present disclosure are based on the discovery that when macrotroponin is present in a biological sample it recognizes and/or specifically binds to an epitope between amino acids 51 to 136 of cTnI (See, FIG. 1A). As illustrated in FIG. 1B, assays for cTnI which employ capture and detection specific binding partners (e.g., antibodies) that recognize and/or specifically bind to epitopes in areas other than between amino acids 51-136, generate a detectable signal if any troponin is present in the biological sample, regardless of any interference by macrotroponin. As shown in FIGS. 2 and 3, assays for cTnI which employ capture and/or detection specific binding partners (e.g., antibodies) that recognize and/or specifically bind to epitopes between amino acid 51-136, generate a reduced, detectable signal if troponin and macrotroponin are each present in the biological sample, thus providing a falsely depressed result (See, FIG. 2—Assay 2; FIG. 3—Assay 3). Thus, the methods of the present disclosure utilize these findings to identify and differentiate macrotroponin from troponin in one or more biological samples from a subject.

In one aspect, the methods of the present disclosure involve obtaining one or more biological samples from a subject. In some aspects, the biological sample can be whole blood. In another aspect, the biological sample can be capillary blood. In yet another aspect, the biological sample can be fingerstick blood. In yet another aspect, the biological sample can be plasma. In yet another aspect, the biological sample can be serum. In still another aspect, the biological sample can be urine. In still yet another aspect, the biological sample can be interstitial fluid. In still yet another aspect, the biological sample can be any combinations of whole blood, capillary blood, fingerstick blood, plasma, serum, urine, or interstitial fluid.

The methods of present disclosure utilize small volumes of one or more biological samples. Specifically, the methods of the present disclosure use biological sample volumes of about 500 μL or less. More specifically, the methods of the present disclosure use less than about 500 μL, about 490 μL, about 480 μL, about 470 μL, about 460 μL, about 450 μL, about 440 μL, about 430 μL, about 420 μL, about 410 μL, about 400 μL, about 390 μL, about 380 μL, about 370 μL, about 360 μL, about 350 μL, about 340 μL, about 330 μL, about 320 μL, about 310 μL, about 300 μL, about 290 μL, about 280 μL, about 270 μL, about 260 μL, about 250 μL, about 240 μL, about 230 μL, about 220 μL, about 210 μL, about 200 μL, about 190 μL, about 180 μL, about 170 μL, about 160 μL, about 150 μL, about 140 μL, about 130 μL, about 120 μL, about 110 μL, about 100 μL, about 90 μL, about 80 μL, about 70 μL, about 60 μL, about 50 μL, about 40 μL, about 30 μL, about 20 μL, or about 10 μL of a biological sample from the subject.

In another aspect, the biological sample is obtained from a subject, such as a human subject. In some aspects, the human subject may be a healthy (e.g., normal) subject. In another aspect, the biological sample is obtained from a non-healthy (e.g., non-normal or not normal) subject. For example, in one aspect, the subject may be suffering from or be suspected of suffering from an acute or chronic myocardial injury. In yet another aspect, the subject may be suffering from or be suspected of suffering from a myocardial infarction (e.g., such as a Type I myocardial infarction or a Type II myocardial infarction). In yet another aspect, the subject may be suffering from or be suspected of suffering from myocardial re-infarction. In still yet another aspect, the subject may be suffering from or be suspected of suffering from acute coronary syndrome (ACS). In yet other aspect, the subject may be suffering from or be suspected of suffering from acute pulmonary embolism. In yet still other aspect, the subject may be suffering from or be suspected of suffering from coronary artery disease (with or without congestive heart failure). In still yet a further aspect, the subject may be suffering from or be suspected of suffering congestive heart failure, such as, for example, stable heart failure. In still yet further aspect, the subject may be suffering from or be suspected of suffering from chronic kidney disease or end-stage renal disease.

In yet another aspect, the methods of present disclosure involve providing a first amount of cardiac troponin (such as cTnI or cTnT) from one or more assays on a biological sample obtained from a subject (hereinafter referred to as a “first assay result” or “assay result 1”). Any suitable assay known in the art can be used, including commercially available cardiac troponin assays. Examples of such assays include, but are not limited to, an immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), a competitive inhibition immunoassay (e.g., forward and reverse), a fluorescence polarization immunoassay (FPIA), an enzyme multiplied immunoassay technique (EMIT), a bioluminescence resonance energy transfer (BRET), and a homogeneous chemiluminescent assay, an one-step antibody detection assay, a homogeneous assay, a heterogeneous assay, a capture on the fly assay, a single molecule detection assay, a clinical chemistry assay, a point-of-care assay, a lateral flow assay, or any combinations thereof. In some aspects, methods for determining the amount of cardiac troponins, such as cardiac troponins I and T (cTnI and cTnT), can be those described in WO 2010099079 and U.S. Pat. Nos. 8,535,895 and 7,838,250 all of which are herein incorporated by reference in their entireties.

In some aspects, the assay used to provide the first assay result is an assay for determining the amount of cTnI in the biological sample. In other aspects, the assay used to provide the first assay result, is an assay for determining the amount of cTnT in the biological sample. In yet other aspects, the assay used to provide the first assay result is a high-sensitivity assay such as a high-sensitivity cTnI assay or a high-sensitivity cTnT assay.

In still further aspects, when the assay used to provide the first assay result is an assay for determining the amount of cTnI in the biological sample, the assay can employ at least one first specific binding partner (e.g., capture reagent) that recognizes and/or specifically binds to epitope between amino acids 1 to 50 or amino acids 137 to 209 of human cTnI. For example, in some aspects, the at least one first specific binding partner (e.g., capture reagent) that recognizes and/or specifically binds an epitope listed below in Table 1.

TABLE 1
Example epitopes recognized by the first specific
binding partner (e.g, capture antibody)

| | Epitope (amino acid) recognized by first |
|---|---|
| | capture reagent (e.g., capture antibody) |
| 1 | 1-15 |
| 2 | 15-25 |
| 3 | 23-29 |
| 4 | 24-40 |
| 5 | 26-35 |
| 6 | 27-40 |
| 7 | 29-34 |
| 8 | 30-35 |
| 9 | 34-37 |
| 10 | 41-49 |
| 11 | 41-50 |
| 12 | 137-148 |
| 13 | 143-152 |
| 14 | 163-209 |
| 15 | 169-178 |
| 16 | 171-178 |
| 17 | 171-190 |
| 18 | 182-191 |
| 19 | 190-196 |
| 20 | 195-209 |

In yet further aspects, the epitope between amino acids 1 to 50 or 137 to 209 of human cTnI has a length of 4 to 50 amino acids. In other aspects, the epitope has a length of 5 to 50 amino acids, 5 to 40 amino acids, 5 to 30 amino acids, 5 to 25 amino acids, 5 to 20 amino acids or 5 to 15 amino acids. In still further aspects, the epitope has a length of 10 to 50 amino acids, 10 to 40 amino acids, 10 to 30 amino acids, or 10 to 25 amino acids. In still other aspects, the epitope has a length of 4 amino acids. In other aspects, the epitope has a length of 5 amino acids. In still other aspects, the epitope has a length of 6 amino acids. In still further aspects, the epitope has a length of 7 amino acids. In yet further aspects, the epitope has a length of 8 amino acids. In still further aspects, the epitope has a length of 9 amino acids. In still further aspects, the epitope has a length of 10 amino acids. In still further aspects, the epitope has a length of 11 amino acids. In yet further aspects, the epitope has a length of 12 amino acids. In still further aspects, the epitope has a length of about 13 amino acids. In still yet further aspects, the epitope has a length of 14 amino acids. In still further aspects, the epitope has a length of 15 amino acids. In still further aspects, the epitope has a length of 16 amino acids. In still further aspects, the epitope has a length of 17 amino acids. In still further aspects, the epitope has a length of 18 amino acids. In yet further aspects, the epitope has a length of 19 amino acids. In still yet further aspects, the epitope has a length of 20 amino acids. In still further aspects, the epitope has a length of 21 amino acids. In yet further aspects, the epitope has a length of 22 amino acids. In yet further aspects, the epitope has a length of 23 amino acids. In still further aspects, the epitope has a length of 24 amino acids. In still further aspects, the epitope has a length of 25 amino acids. In still yet further aspects, the epitope has a length of 26 amino acids. In yet still further aspects, the epitope has a length of 27 amino acids. In still further aspects, the epitope has a length of 28 amino acids. In yet further aspects, the epitope has a length of 29 amino acids. In still further aspects, the epitope has a length of 30 amino acids. In still further aspects, the epitope has a length of 31 amino acids. In yet further aspects, the epitope has a length of 32 amino acids. In yet further aspects, the epitope has a length of 33 amino acids. In still further aspects, the epitope has a length of 34 amino acids. In still further aspects, the epitope has a length of 35 amino acids. In still yet further aspects, the epitope has a length of 36 amino acids. In yet still further aspects, the epitope has a length of 37 amino acids. In still further aspects, the epitope has a length of 38 amino acids. In yet further aspects, the epitope has a length of 39 amino acids. In still further aspects, the epitope has a length of 40 amino acids. In still further aspects, the epitope has a length of 41 amino acids. In yet further aspects, the epitope has a length of 42 amino acids. In yet further aspects, the epitope has a length of 43 amino acids. In still further aspects, the epitope has a length of 44 amino acids. In still further aspects, the epitope has a length of 45 amino acids. In still yet further aspects, the epitope has a length of 46 amino acids. In yet still further aspects, the epitope has a length of 47 amino acids. In still further aspects, the epitope has a length of 48 amino acids. In yet further aspects, the epitope has a length of 49 amino acids. In still further aspects, the epitope has a length of 50 amino acids.

More specifically, in some aspects, the at least one first specific finding partner (e.g., capture reagent) recognizes and/or specifically binds to an epitope between amino acids 24 to 40 on cTnI. Any antibody or antibody fragment that recognizes and/or specifically binds to epitopes 24 to 40 on cTnI can be used as the first specific binding partner (e.g., capture antibody). For example, mouse monoclonal antibodies 1017cc and/or 1039cc and/or rabbit antibodies RecR1, RecR23, RecR33, and/or RecR85, available from HyTest (Turku, Finland), can be used. Additionally, or alternatively, mouse monoclonal antibody TPC100 from SDIX, LLC (Newark, Delaware, U.S.A.) can also be used. If a second specific binding partner (e.g., detection or conjugate reagent) is used in the assay, any specific binding partner can be used (e.g., such as antibody conjugated to a one or more detectable labels) and the second specific binding partner to be used can be determined using routine techniques known in the art.

In still further aspects, when the assay used to provide the first assay result is an assay for determining the amount of cTnT in the biological sample, the assay can employ at least one first specific binding partner (e.g., capture reagent) that recognizes and/or specifically binds to epitope between amino acids 1 to 215 or amino acids 250 to 287 of human cTnT. For example, in some aspects, the at least one first specific binding partner (e.g., capture reagent) that recognizes and/or specifically binds an epitope of amino acids 125-131 of cTnT or 136-147 of cTnT. If a second specific binding partner (e.g., detection or conjugate reagent) is used in the assay, any specific binding partner can be used (e.g., such as antibody conjugated to a one or more detectable labels) and the second specific binding partner to be used can be determined using routine techniques known in the art.

In yet further aspects, the epitope between amino acids 1 to 215 or 250 to 287 of human cTnT has a length of 4 to 50 amino acids. In other aspects, the epitope has a length of 5 to 50 amino acids, 5 to 40 amino acids, 5 to 30 amino acids, 5 to 25 amino acids, 5 to 20 amino acids or 5 to 15 amino acids. In still further aspects, the epitope has a length of 10 to 50 amino acids, 10 to 40 amino acids, 10 to 30 amino acids, or 10 to 25 amino acids. In still other aspects, the epitope has a length of 4 amino acids. In other aspects, the epitope has a length of 5 amino acids. In still other aspects, the epitope has a length of 6 amino acids. In still further aspects, the epitope has a length of 7 amino acids. In yet further aspects, the epitope has a length of 8 amino acids. In still further aspects, the epitope has a length of 9 amino acids. In still further aspects, the epitope has a length of 10 amino acids. In still further aspects, the epitope has a length of 11 amino acids. In yet further aspects, the epitope has a length of 12 amino acids. In still further aspects, the epitope has a length of about 13 amino acids. In still yet further aspects, the epitope has a length of 14 amino acids. In still further aspects, the epitope has a length of 15 amino acids. In still further aspects, the epitope has a length of 16 amino acids. In still further aspects, the epitope has a length of 17 amino acids. In still further aspects, the epitope has a length of 18 amino acids. In yet further aspects, the epitope has a length of 19 amino acids. In still yet further aspects, the epitope has a length of 20 amino acids. In still further aspects, the epitope has a length of 21 amino acids. In yet further aspects, the epitope has a length of 22 amino acids. In yet further aspects, the epitope has a length of 23 amino acids. In still further aspects, the epitope has a length of 24 amino acids. In still further aspects, the epitope has a length of 25 amino acids. In still yet further aspects, the epitope has a length of 26 amino acids. In yet still further aspects, the epitope has a length of 27 amino acids. In still further aspects, the epitope has a length of 28 amino acids. In yet further aspects, the epitope has a length of 29 amino acids. In still further aspects, the epitope has a length of 30 amino acids. In still further aspects, the epitope has a length of 31 amino acids. In yet further aspects, the epitope has a length of 32 amino acids. In yet further aspects, the epitope has a length of 33 amino acids. In still further aspects, the epitope has a length of 34 amino acids. In still further aspects, the epitope has a length of 35 amino acids. In still yet further aspects, the epitope has a length of 36 amino acids. In yet still further aspects, the epitope has a length of 37 amino acids. In still further aspects, the epitope has a length of 38 amino acids. In yet further aspects, the epitope has a length of 39 amino acids. In still further aspects, the epitope has a length of 40 amino acids. In still further aspects, the epitope has a length of 41 amino acids. In yet further aspects, the epitope has a length of 42 amino acids. In yet further aspects, the epitope has a length of 43 amino acids. In still further aspects, the epitope has a length of 44 amino acids. In still further aspects, the epitope has a length of 45 amino acids. In still yet further aspects, the epitope has a length of 46 amino acids. In yet still further aspects, the epitope has a length of 47 amino acids. In still further aspects, the epitope has a length of 48 amino acids. In yet further aspects, the epitope has a length of 49 amino acids. In still further aspects, the epitope has a length of 50 amino acids.

In addition to providing a first assay result, the methods of the present disclosure further involve providing a second amount of cardiac troponin (such as cTnI or cTnT) from one or more assays on the same biological sample obtained from a subject (a “second assay result” or “assay result 2”). Any suitable assay known in the art can be used, including commercially available cardiac troponin assays. Examples of such assays include, but are not limited to, an immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), a competitive inhibition immunoassay (e.g., forward and reverse), a fluorescence polarization immunoassay (FPIA), an enzyme multiplied immunoassay technique (EMIT), a bioluminescence resonance energy transfer (BRET), and a homogeneous chemiluminescent assay, an one-step antibody detection assay, a homogeneous assay, a heterogeneous assay, a capture on the fly assay, a single molecule detection assay, a clinical chemistry assay, a point-of-care assay, a lateral flow assay, or any combinations thereof. In some aspects, methods for determining the amount of cardiac troponins, such as cardiac troponins I and T (cTnI and cTnT), can be those described in WO 2010/099079 and U.S. Pat. Nos. 8,535,895 and 8,8325,120 all of which are herein incorporated by reference in their entireties.

In some aspects, the assay used to provide the second assay result is an assay for determining the amount of cTnI in the biological sample. In other aspects, the assay used to provide the second assay result, is an assay for determining the amount of cTnT in the biological sample. In yet other aspects, the assay used to provide the second assay result is a high-sensitivity assay such as a high-sensitivity cTnI assay or a high-sensitivity cTnT assay.

In still further aspects, when the assay used to provide the second assay result is an assay for determining the amount of cTnI in the biological sample, the assay can employ at least one first specific binding partner (e.g., capture reagent) that recognizes and/or specifically binds to between amino acids 51 to 136 of human cTnI. For example, in some aspects, the at least one first specific binding partner (e.g., capture reagent) that recognizes and/or specifically binds to an epitope between amino acids 51 to 135 of cTnI, amino acids 52 to 136 of cTnI, amino acids 52-136 of cTnI, amino acids 60 to 130 of cTnI, amino acids 60 to 120 of cTnI, amino acids 60 to 115 of cTnI, amino acids 60 to 110 of cTnI, amino acids 60 to 100 of cTnI, amino acids 70 to 125 of cTnI, amino acids 75 to 120 of cTnI, amino acids 75 to 115 of cTnI, amino acids 75 to 110 of cTnI, amino acids 75 to 100 of cTnI, amino acids 80 to 130 of cTnI, amino acids 80 to 120 of cTnI, amino acids 80 to 115 of cTnI, amino acids 80 to 110 of cTnI, or amino acids 80 to 110 of cTnI. In yet further aspects, the epitope between amino acids 80 to 100 of human cTnI has a length of 4 to 20 amino acids. In other aspects, the epitope has a length of 5 to 20 amino acids. In yet other aspects, the epitope has a length of about 5 to 15 amino acids. In still further aspects, the epitope has a length of 10 to about 20 amino acids.

In still other aspects, the epitope has a length of 4 amino acids. In other aspects, the epitope has a length of 5 amino acids. In still other aspects, the epitope has a length of 6 amino acids. In still further aspects, the epitope has a length of 7 amino acids. In yet further aspects, the epitope has a length of 8 amino acids. In still further aspects, the epitope has a length of 9 amino acids. In still further aspects, the epitope has a length of 10 amino acids. In still further aspects, the epitope has a length of about 11 amino acids. In yet further aspects, the epitope has a length of about 12 amino acids. In still further aspects, the epitope has a length of about 13 amino acids. In still yet further aspects, the epitope has a length of about 14 amino acids. In still further aspects, the epitope has a length of about 15 amino acids. In still further aspects, the epitope has a length of about 16 amino acids. In still further aspects, the epitope has a length of about 17 amino acids. In still further aspects, the epitope has a length of about 18 amino acids. In yet further aspects, the epitope has a length of about 19 amino acids. In still yet further aspects, the epitope has a length of about 20 amino acids.

More specifically, in some further aspects, the at least one first specific finding partner (e.g., capture reagent) that recognizes and/or specifically binds to an epitope between amino acids 87 to 92 on cTnI. Any antibody or antibody fragment that recognizes and/or specifically binds to epitopes 87 to 92 on cTnI can be used as the first specific binding partner (e.g., capture antibody). For example, mouse monoclonal antibodies 8E10cc, 560cc or 16A11cc from Hytest ((Turku, Finland),.) can be used. If a second specific binding partner (e.g., detection or conjugate reagent) is used in the assay, any specific binding partner can be used (e.g., such as antibody conjugated to a one or more detectable labels) and the second specific binding partner to be used can be determined using routine techniques known in the art.

In still further aspects, when the assay used to provide the second assay result is an assay for determining the amount of cTnT in the biological sample, the assay can employ at least one first specific binding partner (e.g., capture reagent) that recognizes and/or specifically binds to between amino acids 215 to 250 of human cTnT. For example, in some aspects, the at least one first specific binding partner (e.g., capture reagent) that recognizes and/or specifically binds to an epitope between amino acids 215 to 249 of cTnT, amino acids 216 to 250 of cTnT, amino acids 216-249 of cTnT, amino acids 220 to 245 of cTnT, or amino acids 223 to 242 of cTnT. In yet further aspects, the epitope between amino acids 215 to 250 of human cTnT has a length of 4 to 35 amino acids. In other aspects, the epitope has a length of 5 to 35 amino acids. In yet other aspects, the epitope has a length of 5 to 30 amino acids, 5 to 25 amino acids, 5 to 20 amino acids, or 5 to 15 amino acids. In still further aspects, the epitope has a length of 10 to 35 amino acids, 10 to 30 amino acids, 10 to 25 amino acids, or 10 to 20 amino acids. In still other aspects, the epitope has a length of 4 amino acids.

In other aspects, the epitope has a length of 5 amino acids. In still other aspects, the epitope has a length of 6 amino acids. In still further aspects, the epitope has a length of 7 amino acids. In yet further aspects, the epitope has a length of 8 amino acids. In still further aspects, the epitope has a length of 9 amino acids. In still further aspects, the epitope has a length of 10 amino acids. In still further aspects, the epitope has a length of about 11 amino acids. In yet further aspects, the epitope has a length of about 12 amino acids. In still further aspects, the epitope has a length of about 13 amino acids. In still yet further aspects, the epitope has a length of about 14 amino acids. In still further aspects, the epitope has a length of about 15 amino acids. In still further aspects, the epitope has a length of about 16 amino acids. In still further aspects, the epitope has a length of about 17 amino acids. In still further aspects, the epitope has a length of about 18 amino acids. In yet further aspects, the epitope has a length of about 19 amino acids. In still yet further aspects, the epitope has a length of about 20 amino acids. In still further aspects, the epitope has a length of 21 amino acids. In yet further aspects, the epitope has a length of 22 amino acids. In yet further aspects, the epitope has a length of 23 amino acids. In still further aspects, the epitope has a length of 24 amino acids. In still further aspects, the epitope has a length of 25 amino acids. In still yet further aspects, the epitope has a length of 26 amino acids. In yet still further aspects, the epitope has a length of 27 amino acids. In still further aspects, the epitope has a length of 28 amino acids. In yet further aspects, the epitope has a length of 29 amino acids. In still further aspects, the epitope has a length of 30 amino acids. In still further aspects, the epitope has a length of 31 amino acids. In yet further aspects, the epitope has a length of 32 amino acids. In yet further aspects, the epitope has a length of 33 amino acids. In still further aspects, the epitope has a length of 34 amino acids. In still further aspects, the epitope has a length of 35 amino acids. If a second specific binding partner (e.g., detection or conjugate reagent) is used in the assay, any specific binding partner can be used (e.g., such as antibody conjugated to a one or more detectable labels) and the second specific binding partner to be used can be determined using routine techniques known in the art.

In still further aspects, the assays used to provide the first assay result and the second assay results may be the same type of assays. For example, the assays can each be an immunoassay. Alternatively, the assays used to provide the first assay result and the second assay results can be different types of assays. For example, the assay that provides the first assay result may be an immunoassay and the assay that provides the second assay result may be a clinical chemistry assay. Moreover, the assays used to provide the first and second assay results can be performed simultaneously or sequentially, in any order. In addition, the assays can be performed within a few minutes of each other or within a few hours of each other. Additionally, the order in which the assay results, namely the first assay result and the second assay result are provided is not critical. For example, the first assay result can be provided first followed by the second assay result. Alternatively, the second assay result can be provided first followed by the first assay result. Alternatively, both the first and second assay results can be provided simultaneously, at the same time.

In some aspects, the assay used to provide the first assay result is a high-sensitivity assay for determining the amount of cTnI in the biological sample that employs at least one antibody or antibody fragment thereof, that recognizes and/or specifically binds to epitopes 24 to 40 on cTnI, and the assay used to provide the second assay result is a high-sensitivity assay for determining the amount of cTnI in the biological sample that employs at least one antibody or antibody fragment thereof, that recognizes and/or specifically binds to epitopes 87 to 91 on cTnI. In this aspect, the first assay result is between about 10 ng/L to about 600 ng/L, about 10 ng/L to about 500 ng/L, about 10 ng/L to about 400 ng/L, about 10 ng/L to about 300 ng/L, about 10 ng/L to about 200 ng/L, about 10 ng/L to about 100 ng/L, about 10 ng/L to about 75 ng/L, about 10 ng/L to about 50 ng/L, about 10 ng/to about 40 ng/L, about 10 ng/L to about 30 ng/L, about 10 ng/L to about 20 ng/L, about 20 ng/L to about 600 ng/L, about 20 ng/L to about 500 ng/L, about 20 ng/L to about 400 ng/L, about 20 ng/L to about 300 ng/L, about 20 ng/L to about 200 ng/L, about 20 ng/L to about 100 ng/L, about 20 ng/L to about 75 ng/L, about 20 ng/L to about 50 ng/L, about 20 ng/to about 40 ng/L, about 20 ng/L to about 30 ng/L, about 30 ng/L to about 600 ng/L, about 30 ng/L to about 500 ng/L, about 30 ng/L to about 400 ng/L, about 30 ng/L to about 300 ng/L, about 30 ng/L to about 200 ng/L, about 30 ng/L to about 100 ng/L, about 30 ng/L to about 75 ng/L, about 30 ng/L to about 50 ng/L, about 30 ng/to about 40 ng/L, about 40 ng/L to about 600 ng/L, about 40 ng/L to about 500 ng/L, about 40 ng/L to about 400 ng/L, about 40 ng/L to about 300 ng/L, about 40 ng/L to about 200 ng/L, about 40 ng/L to about 100 ng/L, about 40 ng/L to about 75 ng/L, about 40 ng/L to about 50 ng/L, about 50 ng/L to about 600 ng/L, about 50 ng/L to about 500 ng/L, about 50 ng/L to about 400 ng/L, about 50 ng/L to about 300 ng/L, about 50 ng/L to about 200 ng/L, about 50 ng/L to about 100 ng/L, or about 50 ng/L to about 75 ng/L.

Once the first assay result and second assay results from the biological sample are obtained, a percentage difference or ratio of the cardiac troponin in each of the assays is calculated using one of the following formulas:

$$Percentage difference : ( 1 - ( second assay result / first assay result ) ) \times 100 \%$$

Ratio: (a) 1−(second assay result/first assay result); or
(b) second assay result/first assay result.

Any of the above formulas can be used in the methods described herein. Once the percentage difference or ratio between the assay results is calculated, a determination can be made whether the biological sample contains any macrotroponin (e.g., to differentiate any macrotroponin from troponin in the biological sample). Specifically, when the percentage difference is calculated, macrotroponin is determined to be present in the biological sample if the percentage difference is less than about −15% or greater than about 45%. In other aspects, macrotroponin is determined to be present in the biological sample if the percentage difference is less than −20% or greater than about 50%. By way of example, if the percentage difference is determined to be 5%, then macrotroponin would be determined not to be present in the biological sample. In contrast, if the percentage difference is determined to 50%, then macrotroponin would be determined to be present in the biological sample.

Alternatively, when a ratio is calculated using the formula: 1−(second assay result/first assay result), macrotroponin is determined to be present in the biological sample if the percentage difference is less than about −0.15 or greater than about 0.45. In other aspects, macrotroponin is determined to be present in the biological sample if the percentage difference is less than −0.20 or greater than about 0.50. By way of example, if the ratio is determined to be 0.05, then macrotroponin would not be determined to be present in the biological sample. In contrast, if the ratio is determined to 0.50, then macrotroponin would be determined to be present in the biological sample.

Alternatively, when a ratio is calculated using the formula: second assay result/first assay result, macrotroponin is determined to be present in the biological sample if the percentage difference is less than about 0.85 or greater than about 1.45. In other aspects, macrotroponin is determined to be present in the biological sample if the percentage difference is less than 0.80 or greater than about 1.50. By way of example, if the ratio is determined to be 1.05, then macrotroponin would not be determined to be present in the biological sample. In contrast, if the ratio is determined to 1.50, then macrotroponin would be determined to be present in the biological sample.

The methods of the present disclosure are not limited by the method of obtaining the first assay result and second assay result. In some embodiments, the methods comprise receiving the first and/or second assay results from a testing lab, from said subject, from an analytical testing system, and/or from a hand-held or point-of-care testing device (e.g., using a point-of-care assay). In some aspects, the methods comprise obtaining the first and/or second assay results electronically.

Determining the concentration of cardiac troponin (e.g., cTnI or cTnT) by the one or more assays described herein can be adapted for use in a variety of automated and semi-automated systems or platforms (including those wherein the solid phase comprises a microparticle) known in the art. The following adaptations of automated and/or semi-automated systems are included herein as merely exemplary. Specifically, the methods can utilize automated and semi-automated systems or platforms such as those described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, IL) as Abbott Point of Care (i-STAT® or i-STAT Alinity, ID Now®, Abbott Laboratories) as well as those described in U.S. Pat. Nos. 5,089,424 and 5,006,309, and as commercially marketed, e.g., by Abbott Laboratories (Abbott Park, IL) as ARCHITECT® or the series of Abbott Alinity devices.

Other methods of detection include the use of or can be adapted for use on a nanopore device or nanowell device, e.g., for single molecule detection. As used herein the term “single molecule detection” refers to the detection and/or measurement of a single molecule of an analyte in a test sample at very low levels of concentration (such as pg/mL or femtogram/mL levels). A number of different single molecule analyzers or devices are known in the art and include nanopore and nanowell devices. Examples of nanopore devices are described in PCT International Application WO 2016/161402, which is hereby incorporated by reference in its entirety. Examples of nanowell device are described in PCT International Application WO 2016/161400, which is hereby incorporated by reference in its entirety.

### EXAMPLES

The following examples are for purposes of illustration only and are not intended to limit the scope of the claims.

### Example 1

Serum and plasma samples were obtained from normal human subjects (blood donors) and subjects reporting to the emergency department and diagnosed as having a myocardial infarction (MI). A first immunoassay was performed to measure the amount of human cTnI in the samples. The first immunoassay (Assay 1) used as a capture antibody an antibody that recognizes amino acids 24 to 40 on cTnI. The assay was performed according to the manufacturer's instructions. A second immunoassay was performed to measure the amount of human cTnI in the same biological samples obtained from the subjects. The second immunoassay (Assay 2) used as a capture antibody that recognizes amino acids 87-91 on cTnI. The assay was performed according to manufacturer's instructions. The results from two manual methods to identify the presence of macrotroponin, PEG precipitation and Size Exclusion Chromatography (SEC), in addition to the results from assays 1 and 2 are shown in the table below. The PEG method consists of splitting a sample into 2 aliquots, 200 uL each. The 200 μL of PBS is added to the first aliquot to serve as a control and 200 μL of a 25% 8000 MW PEG/PBS solution is added to the second aliquot. Both samples are mixed, incubated at 10 minutes at room temperature and then centrifuged at an RCF of 10,000×g for 10 minutes. The supernatant is transferred to a new sample cup and tested with the cTnI assay of choice. In this instance it was the ARCHITECT hsTnI assay. The results obtained between the 2 aliquots are compared and a % difference is determined. Percent recoveries of less than 26% are indicative of the presence of macrotroponin using the PEG method. The SEC method consists of loading 100 μL of sample onto a TSKgel SWxl guard column and super SW3000 SEC column. The sample is then eluted with PBS at a rate of 0.3 mL/min. Fractions are collected every minute and then tested in the cTnI assay of choice. In this instance it was the ARCHITECT hsTnI assay. The presence of a peak or secondary peak between fractions 9 and 13 is indicative of the presence of macrotroponin using the SEC method. Unbound troponin I is detected between fractions ~14-16, with the peak at fraction 15. The results are sorted by the % macrodifferentiation result, from lowest to highest bias observed. The SEC method is indicating the presence of macrotroponin when the % macrodifferentiation result is ≤−15% and ≥52%. The PEG method is indicating the presence of macrotroponin when the % macrodifferentiation result is ≥52%. A negative % macrodifferentiation result is indicative of the presence of macrotroponin interfering with assay 2 and a positive % macrodifferentiation result is indicative of the presence of macrotroponin interfering with assay 1.

| | | Manual, PEG | | | | | | | |
|---|---|---|---|---|---|---|---|---|---|
| | | Precipitation | | | Manual, Size Exclusion Chromatography | | | | |
| | | Assay 1 | | | Fraction Number | | | | |
| | | Epitope | | % Recovery | (Secondary Peak Between Fractions | | | | |
| | | 24-40 | | (< [symbol] % | 9-13 [symbol] ndicative of Macro [symbol] | | | | |
| | | (ng/L) | | indicative of | 9 | | | 12 | 13 |
| Category | [symbol] ID | Control | PEG | MacroT [symbol] | [symbol] | 10 | 11 | [symbol] | |
| MI | Subject 1 | [symbol] | 79.1 | 43% | 6.9 | 23.7 | 9.4 | 7.4 | 13.7 |
| MI | Subject 2 | 17.7 | 4.7 | 2 [symbol] % | 0.0 | 2.0 | 1.5 | 1.0 | 0.5 |
| MI | Subject 3 | 177.9 | [symbol] .7 | 30% | 1.7 | 16.0 | [symbol] | 9. [symbol] | 17.1 |
| MI | Subject 4 | [symbol] .2 | [symbol] | 42% | 0.7 | 31. [symbol] | 9.6 | 7.9 | 15.3 |
| MI | Subject 5 | 29.1 | 9.1 | 31% | 0.0 | 2.9 | 1. [symbol] | 0.5 | 1.0 |
| MI | Subject 6 | [symbol] | 66.3 | 34% | 0.2 | 2.1 | 2.7 | 7.2 | 14.7 |
| Donor | Subject 7 | [symbol] | 12. [symbol] | 25% | 3.4 | 2.7 | 2.3 | 2.2 | 3.7 |
| MI | Subject 8 | 93.0 | 19. [symbol] | 21% | 0.6 | 1.4 | 0.6 | 0.9 | 2.2 |
| MI | Subject 9 | [symbol] .3 | 205.1 | 37% | 0.1 | 3.4 | 4. [symbol] | [symbol] .1 | 16.5 |
| MI | Subject 10 | [symbol] | 64.6 | 20% | 0.6 | 2.4 | 1. [symbol] | 4.3 | 24.4 |
| MI | Subject 11 | [symbol] | 99.9 | [symbol] % | 6.4 | 2. [symbol] | 3.2 | 5.5 | 10.4 |
| MI | Subject 12 | 137.9 | [symbol] 4.8 | 40% | 1.3 | 3.0 | 3.5 | 6.1 | [symbol] .5 |
| MI | Subject 13 | 601.2 | 197.3 | 3 [symbol] % | 2.0 | 4.5 | 6.6 | [symbol] .7 | 22.5 |
| MI | Subject 14 | 5 [symbol] | 0. [symbol] | 15% | 0.7 | 2.7 | 2.1 | 0.7 | 1.1 |
| MI | Subject 15 | [symbol] .4 | [symbol] | 3 [symbol] % | 0.2 | 4.3 | 4.9 | 7.6 | 19.3 |
| MI | Subject 16 | 225.0 | 103. [symbol] | [symbol] % | 0.3 | 1.9 | 2.4 | 3.3 | 7. [symbol] |
| MI | Subject 17 | 291.7 | [symbol] | 30% | 6.5 | 5.8 | 11.7 | 11.7 | 17.1 |
| MI | Subject 18 | 2534.1 | [symbol] .4 | 35% | 0.6 | 6.2 | 13.7 | 25.2 | 52.9 |
| MI | Subject 19 | 19.8 | 5.1 | 20% | 0 | 2. [symbol] | [symbol] | 0.1 | 1.8 |
| MI | Subject 20 | 56.1 | 0.5 | 1% | 0. [symbol] | 3. [symbol] | 22.3 | 21.5 | [symbol] .7 |
| Donor | Subject 21 | 44.1 | 0.3 | 1% | 2.5 | 5.2 | 6.5 | 6.4 | 3.7 |
| MI | Subject 22 | 45.1 | 0.2 | 0% | 17 | 4.9 | 16.2 | 25.2 | [symbol] .1 |
| Donor | Subject 23 | [symbol] | 1. [symbol] | 10% | 2.2 | 3.1 | 2.8 | 7.9 | [symbol] |
| MI | Subject 24 | [symbol] .4 | 2.4 | 26% | 0.7 | 1.6 | 1.5 | 0. [symbol] | 1.3 |
| Donor | Subject 25 | 45.2 | 0.6 | 1% | 3.9 | 3 | 4.7 | 10.5 | 5.2 |
| Donor | Subject 26 | 38.7 | 3.3 | 9% | 3.2 | 4.7 | 7.2 | 7.7 | 5.4 |
| Donor | Subject 27 | [symbol] .5 | 2.6 | 2% | 3.1 | 9.5 | 23 | 25.6 | 8.9 |

| | | Manual, Size Exclusion Chromatography | | | | | Automated, Macro- | | |
|---|---|---|---|---|---|---|---|---|---|
| | | Fraction Number | | | | | differentiation Assay | | |
| | | (Secondary Peak Between Fractions | | | | | Assay 1 | Assay 2 | % Macro- |
| | | 9-13 indicative of Macro [symbol] ) | | | | | Epitope | Epitope | differ- |
| | | 14 | | 16 | | 18 | 24-40 | 87-91 | entiation |
| Category | [symbol] ID | [symbol] | 15 | [symbol] | 17 | [symbol] | (ng/L) | (ng/L) | Results |
| MI | Subject 1 | 32.4 | 49.4 | 21.7 | 4.7 | 0.7 | 500.5 | 643.5 | −29% |
| MI | Subject 2 | 3.3 | 5.6 | 4.4 | 0.7 | 0.0 | [symbol] .7 | 41.1 | −22% |
| MI | Subject 3 | 34.1 | 51.9 | 21.1 | 4.1 | 0. [symbol] | 434.7 | 524.9 | −21% |
| MI | Subject 4 | 31.5 | 58.5 | 22.3 | 4.7 | 1.4 | 512.2 | 610. [symbol] | −19% |
| MI | Subject 5 | [symbol] .3 | 7.8 | 4.6 | 0. [symbol] | 0.5 | 44.5 | 51.2 | −15% |
| MI | Subject 6 | 41.7 | 72.2 | 36 | [symbol] | 1.3 | 341.9 | 385.9 | −13% |
| Donor | Subject 7 | 6.7 | 12.9 | 7.2 | 4 | 3.2 | 57.0 | 62.3 | [symbol] % |
| MI | Subject 8 | 10.1 | 15.6 | 7.6 | 1.2 | 0.4 | 184. [symbol] | 192.5 | −4% |
| MI | Subject 9 | 117.2 | 164.8 | 62.1 | 11.6 | 1.8 | 923.4 | 922.1 | 0% |
| MI | Subject 10 | 72 | [symbol] .4 | 45.1 | 10.1 | 1.9 | [symbol] | [symbol] .0 | 1% |
| MI | Subject 11 | 46 | [symbol] | [symbol] | 10.2 | 2.4 | [symbol] | [symbol] | 4% |
| MI | Subject 12 | 26.5 | [symbol] .9 | 21 | 5.7 | 1.4 | 41 [symbol] .3 | [symbol] .1 | 14% |
| MI | Subject 13 | 117.7 | 167 | 62.8 | 12.1 | 2.2 | 14 [symbol] .0 | [symbol] | 19% |
| MI | Subject 14 | 0.9 | 1.6 | 1.9 | 1.1 | 1.1 | 21.9 | 17.1 | 22% |
| MI | Subject 15 | 54 | 139.6 | 77. [symbol] | 16.9 | 2.7 | 1534.2 | 1132.1 | 2 [symbol] % |
| MI | Subject 16 | 45 | 73.4 | 36. [symbol] | 10 | 1.9 | [symbol] .2 | 4 [symbol] .4 | 2 [symbol] % |
| MI | Subject 17 | 67.8 | 81.2 | 56 | [symbol] .2 | 6.2 | [symbol] | 600.3 | 32% |
| MI | Subject 18 | 348.3 | [symbol] .1 | 403 | 84.4 | 12.6 | 7 [symbol] .1 | 4 [symbol] .9 | 42% |
| MI | Subject 19 | 4.2 | 7.4 | 4.5 | 1.1 | 1.5 | 22.4 | 10. [symbol] | 52% |
| MI | Subject 20 | 2.9 | 1.6 | 0.9 | 0.5 | 0. [symbol] | 119.7 | 4 [symbol] .3 | 60% |
| Donor | Subject 21 | 2.2 | 1.9 | 1. [symbol] | 1.2 | 1.5 | 54. [symbol] | [symbol] | 62% |
| MI | Subject 22 | 3.1 | 2. [symbol] | 0.3 | 0.7 | 0.9 | 178.7 | 5 [symbol] .6 | [symbol] % |
| Donor | Subject 23 | 2.4 | 3.3 | 1.6 | 1.8 | 1. [symbol] | 32.5 | 4.0 | [symbol] % |
| MI | Subject 24 | 1.8 | 4.5 | 2.2 | 0.6 | 0.4 | 21.3 | 2. [symbol] | [symbol] % |
| Donor | Subject 25 | 2.8 | 2.7 | 2. [symbol] | 3 | 1.5 | 87.6 | 7.6 | 91% |
| Donor | Subject 26 | 3.1 | 3 | 1.9 | 1.9 | 2.1 | [symbol] .6 | [symbol] | 92% |
| Donor | Subject 27 | 5.6 | 4.3 | 3 | 2.7 | 2.2 | [symbol] .4 | [symbol] .2 | 96% |

[symbol]

indicates data missing or illegible when filed

Although only a few exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

1. A method for identifying the presence of macrotroponin in a biological sample obtained from a subject, the method comprising the steps of:
a) providing a first amount of cardiac troponin I (cTnI) obtained from an assay on a biological sample obtained from a subject (“assay result 1”), wherein the assay employs at least one antibody or antibody fragment that recognizes or specifically binds to an epitope between amino acids 1 to 50 or 137 to 209 on cTnI;
b) providing a second amount of cardiac troponin I (cTnI) obtained from an assay from the same biological sample from the subject (“assay result 2”), wherein the assay employs at least one antibody or antibody fragment that recognizes or specifically binds to an epitope between amino acids 51 to 136 on cTnI;
c) determining a percentage difference of cTnI in the sample using the formula: (1−(assay result 2/assay result 1))×100%; and
d) determining that macrotroponin is present in the sample if the percentage difference determined in step c) is less than about −15% or greater than about 45%.

2. The method of claim 1, wherein the epitope between amino acids 1 to 50 or 137 to 209 has a length of 4 to 50 amino acids.

3. The method of claim 1, wherein the epitope between amino acids 51 to 136 has a length of 4 to 20 amino acids.

4. The method of claim 1, wherein the assay employed to provide assay result 1 employs at least one antibody or antibody fragment that recognizes or specifically binds to epitopes 24-40 on cTnI.

5. The method of claim 1, wherein the assay employed to provide assay result 2 employs at least one antibody or antibody fragment that recognizes or specifically binds to epitopes 87 to 91 on cTnI.

6. The method of claim 1, wherein the volume of biological sample used in the method is less than about 500 μL, less than about 400 μL, less than about 300 μL, less than about 200 μL, less than about 100 μL, less than about 90 μL, less than about 80 μL, less than about 70 μL, less than about 60 μL, less than about 50 μL, less than about 40 μL, less than about 30 μL, less than about 20 μL, or less than about 10 μL.

7. The method of claim 1, wherein the assay is: (a) a high-sensitivity cTnI assay; (b) an immunoassay, a clinical chemistry assay, a lateral flow assay, or any combinations thereof; (c) a point-of-care assay or a single molecule detection assay; or (d) any combination of (a)-(c).

8. (canceled)

9. (canceled)

10. The method of claim 1, wherein the biological sample is whole blood, capillary blood, fingerstick blood, plasma, serum, urine, or any combinations thereof.

11. The method of claim 1, wherein the subject is a human.

12. The method of claim 1, wherein macrotroponin is identified as being present in the sample if the percentage difference determined in step c) is less than −15% or greater than about 45%.

13. The method of claim 12, wherein assay result 1 is between about 10 ng/L to about 600 ng/L or about 10 ng/L to about 100 ng/L.

14. (canceled)

15. A method for identifying the presence of macrotroponin in a biological sample obtained from a subject, the method comprising the steps of:
a) providing a first amount of cardiac troponin I (cTnI) obtained from an assay on a biological sample obtained from a subject (“assay result 1”), wherein the assay employs at least one antibody or antibody fragment that recognizes or specifically binds to an epitope between amino acids 1 to 50 or 137 to 209 on cTnI;
b) providing a second amount of cardiac troponin I (cTnI) obtained from an assay from the same biological sample from the subject (“assay result 2”), wherein the assay employs at least one antibody or antibody fragment that recognizes or specifically binds to an epitope between amino acids 51 to 136on cTnI;
c) determining a ratio of cTnI in the sample using the formula: 1−(assay result 2/assay result 1); and
d) determining that macrotroponin is present in the sample if the ratio determined in step c) is less than about −0.15 or greater than about 0.45.

16. The method of claim 15, wherein the epitope between amino acids 1 to 50 or 137 to 209 has a length of 4 to 50 amino acids.

17. The method of claim 15, wherein the epitope between amino acids 51 to 136 has a length of 4 to 20 amino acids.

18. The method of claim 15, wherein the assay employed to provide assay result 1 employs at least one antibody or antibody fragment that recognizes or specifically binds to epitopes 24-40 on cTnI.

19. The method of claim 16, wherein the assay employed to provide assay result 2 employs at least one antibody or antibody fragment that recognizes or specifically binds to epitopes 87 to 91 on cTnI.

20. The method of claim 15, wherein the assay is a high-sensitivity cTnI assay; (b) an immunoassay, a clinical chemistry assay, a lateral flow assay, or any combinations thereof; (c) a point-of-care assay or a single molecule detection assay; or (d) any combination of (a)-(c).

21. (canceled)

22. (canceled)

23. The method of claim 15, wherein the biological sample is whole blood, capillary blood, fingerstick blood, plasma, serum, urine, or any combinations thereof.

24. The method of claim 15, wherein the subject is a human.

25. The method of claim 15, wherein macrotroponin is identified as being present in the sample if the ratio determined in step c) is less than −0.20 or greater than about 0.50.

26. The method of claim 25, wherein assay result 1 is between about 10 ng/L to about 600 ng/L or about 10 ng/L to about 100 ng/L.

27. (canceled)
